(19) United States
(12) Patent Application Publication
(10) Pub. No.: US 2024/0268746 A1
(43) Pub. Date: Aug. 15, 2024

(54) SYSTEM AND METHOD FOR WIRELESS RECORDING OF BRAIN ACTIVITY

(71) Applicant: The Regents of the University of California, Oakland, CA (US)

(72) Inventors: Ahmet Ali Yanik, Santa Cruz, CA (US); Ahsan Habib, Santa Cruz, CA (US); Neil Hardy, Santa Cruz, CA (US)

(21) Appl. No.: 18/568,909

(22) PCT Filed: Jun. 13, 2022
(86) PCT No.: PCT/US2022/033200

§ 371 Date: Dec. 11, 2023

Related U.S. Application Data
(60) Provisional application No. 63/331,409, filed on Apr. 15, 2022.
(60) Provisional application No. 63/209,492, filed on Jun. 11, 2021.

(51) Int. Cl. A61B 5/00

(52) U.S. Cl. CPC A61B 5/4064; A61B 5/0075; A61B 2562/0285

(57) ABSTRACT

A system includes a light source configured to illuminate a target site in a brain with a near infrared light having a wavelength from about 1000 nm to about 1700 nm. The system also includes a plurality of nanoparticle probes disposed at the target site, each of the nanoparticle probes may include: a core having a substantially spherical shape, a conductive shell disposed over the core, and an electrochromic polymer coating disposed over the conductive shell. The system may further include an image sensor configured to receive backscattered light from the plurality of nanoparticles illuminated by the near infrared light. The plurality of nanoparticle probes is configured to shift their backscattering spectrum in response to a change in an electrical field at the target site.

### CROSS-REFERENCE TO RELATED APPLICATION

The present application claims the benefit of and priority to U.S. Provisional Application No. 63/209,492, filed on Jun. 11, 2021; and U.S. Provisional Application No. 63/331,409, filed on Apr. 15, 2022. The entire disclosures of each of the foregoing applications are incorporated by reference herein.

### GOVERNMENT LICENSE RIGHTS

This invention was made with government support under Grant No. 1611290, awarded by Electrical, Communications and Cyber Systems Division of the National Science Foundation. The Government has certain rights in the invention.

### BACKGROUND

Understanding how the human brain performs complex functions is one of the greatest scientific, engineering, and medical challenges of the 21st century. It requires a system level approach unraveling how millions of neurons communicate with each other across different anatomical regions of the brain with varying temporal and spatial characteristics and connection strengths. Recording brain activity with high spatiotemporal resolution and in a massively multiplexed manner remains technologically distant. Advancement of fundamentally new detection mechanisms is needed. Microelectrode arrays (MEAs), the workhorse of neuroscientists, offer multiplexed electrophysiological recordings with high temporal resolution. However, their use is inherently limited to a few hundred electrodes as direct electronic measurements suffer from complex wiring requirements and inherent bandwidth (e.g., spatial multiplexing) limitations due to electron-electron interactions within conductors. Moreover, electrode arrays can only record from small sections of the brain and require invasive cranial surgical operations. Thus, a fundamental limitation for the implantable brain-machine interfaces (BMI) is the wiring requirements for power transfer and signal transmission. Furthermore, existing electrode technologies can only record from localized sections of the brain and require invasive cranial surgeries.

Recent discovery of genetically encoded voltage sensitive fluorescence indicators (GEVI) has created tremendous excitement as light offers unparalleled (e.g., wavelength/time/space division) multiplexing and information carrying capabilities. This technique, on the other hand, cannot be implemented in human studies as it requires genetic incorporation of voltage sensitive molecules to neurons. In addition, strong attenuation of visible light in biological tissue renders much of the brain inaccessible to fluorescence-based techniques. Functional near infrared spectroscopy (fNIRS) overcomes visible light limitations and provides non-invasive (non-surgical) volumetric readout of the brain activity as the near infrared (NIR) light can penetrate through the skull and into the brain cortex. fNIRS, however, can only measure local variations in the cerebral blood volume and oxygenation dynamics, not the electrophysiological signals.

Conventional brain activity monitoring methods, such as intracranial electroencephalogram (iEEG), are used for estimating the epileptogenic cortex in severe epilepsy and require placing intracranial electrodes with either large portions of the skull removed (e.g., electrocorticography (ECoG)) or by drilling multiple burr holes for multiple electrodes (e.g., stereoelectroencephalography (SEEG)). Another downside to iEEG monitoring is that the electrodes can only measure highly localized regions at a time, requiring other methods to be used to narrow the location of the epileptogenic region(s). Most epilepsy patients have drug-resistant epilepsy and only have the option of highly invasive surgical interventions. In the clinic, non-invasive methods are used with limited resolution (e.g., magnetoencephalography (MEG)) and/or to provide indirect indicators of activity (e.g., fMRI) to supplement iEEG. Together, these aid surgical planning, but depending on the surgery approximately 20-60% of patients do not see seizure remission and this does not include those which experience deteriorating cognitions or fatality as a result. Surgical methods are improving, particularly in ablative and radiosurgery, but diagnostic tools to investigate electrical activity have not kept pace; it stands to reason that giving surgeons better tools can better the odds for those with brain diseases.

### SUMMARY

The present disclosure provides system-on-a-nanoparticle probes, which act as neurophotonic solution-dispersible wireless activity reporters and may be used for massively multiplexed measurements, enabling wireless detection of in vivo bioelectrical signals. The nanoparticle probes enable non-invasive measurement of in vivo electro-physiological activity using near-infrared light. In particular, the nanoparticle probe is configured to convert electrophysiological activity to an optically detectable signal that can be picked up from outside the brain using a second window near-infrared (NIR-II, 1000-1700 nm) light reader. Much like the passive radio frequency identification (RFID) tags, the nanoparticle probe reports the spiking activity of cells by modulating the incoming NIR light coupling and the re-radiated light spectrum that is sent back to the reader using backscattering. Here, the spectrum of the backscattered NIR light is modulated by the electrochromic loading of the plasmonic (electro-plasmonic) nanoantenna, which shows strong sensitivity to the local electric-field dynamics. Thus, each of the nanoparticle probes provides a bioelectrical signal detection capability in a single nanoparticle device that includes wireless power, electrophysiological signal detection, and data broadcasting capabilities at nanoscale dimensions.

The nanoparticle probe enables use of infrared light within the biologically transparent near infrared (NIR-II, 1000-1700 nm) window for direct read-out through the skull. The nanoparticle also avoids invasive surgical operations for implanting conventional BMIs since no wiring or power supply is needed for the wireless (electro-plasmonic) excitation and remote detection. In addition, due to diminutive dimensions (i.e., less than 200 nm) of the nanoparticle probe, which is comparable to viral particles, the nanoparticle probe may be delivered through the blood-brain barrier to different regions of the brain without surgical operations. Also, since the nanoparticle probes are much smaller than the critical dimensions that trigger glial cell response (e.g., about 12 μm), the nanoparticle probes offer a long-term operation capability. Electro-plasmonic signal conversion removes front-end signal processing requirements and allows for large scale in vivo measurements that are not restricted by electrode dimensions and wiring, and electronic bandwidth limitations. The nanoparticle probes also provide high signal to shot noise ratio (SSNR of about 10³) recording capability from single neurons due to its enhanced cross-section of about 10⁴ nm² and field-sensitivity of up to approximately 40%.

Nanoparticle probes of the present disclosure open an entirely new practice of electrophysiological investigation of pathologies in the peripheral nervous system (PNS). Currently, medical professionals rely on patients' communicable symptoms and basic electrode tests such as nerve conduction studies (NCS) and needle electromyography (EMG) to diagnose problems in the peripheral nervous system. These use external skin electrodes and, for EMG, a thin needle electrode to determine nervous conduction velocity and muscular response. These methods look at muscular response to diagnose peripheral neuropathies, but neither method can image the peripheral nervous system. The only method attempting to image the PNS thus far is magnetoneurography (MNG). However, MNG requires a prohibitively expensive superconducting quantum interference device biomagnetometer system in a magnetically shielded room and is limited in resolution on the order of millimeters. The nanoparticle probes can be applied topically to target peripheral nerves, which is an ideal scenario for this technology as NIR light can easily penetrate skin and illuminate neural activity.

The disclosed nanoparticle probes technology will open up new horizons for brain-machine interfaces. Integration of NIR nanoparticle probes technology with fNIRS will also allow for an all-optical methodology, that can measure and distinguish the fast neuronal signaling (captured by nanoparticle probes) and the slow hemodynamic activity (measured by fMRI).

According to one embodiment of the present disclosure, a nanoparticle probe for detecting neural activity is disclosed. The nanoparticle includes a core having a substantially spherical shape; a conductive shell disposed over the core, and an electrochromic polymer coating disposed over the conductive shell.

Implementations of the above embodiment may include one or more of the following features. According to one aspect of the above embodiment, the nanoparticle probe may also include a biological coating that may in turn include at least one of lipids, proteins, or peptides. The core may be formed from a dielectric material or a magnetic material. The core may include at least one of silica or magnetite. The core may have a diameter from about 80 nm to about 150 nm. The nanoparticle probe may have a diameter from about 140 nm to about 200 nm. The conductive shell may include at least one of graphene, gold, silver, aluminum, copper, titanium, magnesium, palladium, and zirconium. The electrochromic polymer coating may include at least one of poly(3,4-ethylenedioxythiophene): polystyrene sulfonate, polypyrrole, polyaniline, or poly(3,4-propylenedioxythiophene). The conductive shell may have a thickness from about 1 nm to about 10 nm. The electrochromic polymer coating may have a thickness from about 10 nm to about 30 nm. The nanoparticle probe is configured to exhibit resonance scattering of near infrared light having a wavelength from about 1000 nm to about 1100 nm.

According to another embodiment of the present disclosure, a method for monitoring neural activity is disclosed. The method may include delivering a plurality of nanoparticle probes to a target site within a brain and illuminating the target site with a near infrared light having a wavelength from about 1000 nm to about 1700 nm. The method also includes receiving backscattered light from the plurality of nanoparticles illuminated by the near infrared light and determining neural activity based on the backscattered light from the plurality of nanoparticles.

Implementations of the above embodiment may include one or more of the following features. According to one aspect of the above embodiment, delivering may also include at least one of cerebellomedullary cistern delivery, intracarotid delivery, intraventricular injection, stereotactic brain injection, or intranasal delivery. The plurality of nanoparticle probes is configured to shift their backscattering spectrum in response to a change in an electrical field at the target site. Each nanoparticle probe of the plurality of probes may include: a core having a substantially spherical shape; a conductive shell disposed over the core; and an electrochromic polymer coating disposed over the conductive shell. Each nanoparticle probe of the plurality of probes may further include a biological coating may include at least one of lipids, proteins, or peptides. The biological coating may be integrated with or disposed over the electrochromic polymer. The core may be formed from a magnetic material and delivering may include magnetically guiding the plurality of nanoparticle probes to the target site.

According to a further embodiment of the present disclosure, a system for monitoring neural activity is disclosed. The system includes a light source configured to illuminate a target site in a brain with a near infrared light having a wavelength from about 1000 nm to about 1700 nm. The system also includes a plurality of nanoparticle probes disposed at the target site, each of the nanoparticle probes may include: a core having a substantially spherical shape, a conductive shell disposed over the core, and an electrochromic polymer coating disposed over the conductive shell. The system may further include an image sensor configured to receive backscattered light from the plurality of nanoparticles illuminated by the near infrared light. The plurality of nanoparticle probes is configured to shift their backscattering spectrum in response to a change in an electrical field at the target site.

Implementations of the above embodiment may include one or more of the following features. According to one aspect of the above embodiment, each of the nanoparticle probes may include a biological coating that in turn may include at least one of lipids, proteins, or peptides. The core may be formed from a dielectric material or a magnetic material. The core may include at least one of silica or magnetite. The core may have a diameter from about 80 nm to about 150 nm. The conductive shell may include at least one of graphene, gold, silver, aluminum, copper, titanium, magnesium, palladium, and zirconium. The electrochromic polymer coating may include at least one of poly(3,4-ethylenedioxythiophene): polystyrene sulfonate, polypyrrole, polyaniline, or poly(3,4-propylenedioxythiophene). The conductive shell may have a thickness from about 1 nm to about 10 nm. The electrochromic polymer coating may have a thickness from about 10 nm to about 30 nm. The nanoparticle probe is configured to exhibit resonance scattering of near infrared light having a wavelength from about 1000 nm to about 1100 nm.

### BRIEF DESCRIPTION OF DRAWINGS

Various embodiments of the present disclosure are described herein below with reference to the figures wherein:

FIG. 1 is a schematic, cross-sectional view of a nanoparticle probe according to an embodiment of the present disclosure;

FIG. 2 is a system for delivering the plurality of nanoparticle probe of FIG. 1 in a brain according to an embodiment of the present disclosure;

FIG. 3 is a schematic diagram of a brain with a plurality of nanoparticle probe of FIG. 1 according to an embodiment of the present disclosure;

FIG. 4 is a schematic diagram illustrating shifts in spectrum of the nanoparticle probe in response to depolarization and hyperpolarization of a neuron according to an embodiment of the present disclosure;

FIG. 5 is a flow chart for deploying and using the plurality of nanoparticle probes of FIG. 1 in a brain according to an embodiment of the present disclosure;

FIG. 6 shows optical scattering efficiency plots of the nanoparticle probe of FIG. 1 according to an embodiment of the present disclosure;

FIG. 7 shows a differential scattering signal as a function of a thickness of a coating of the nanoparticle probe of FIG. 1 according to an embodiment of the present disclosure; and

FIG. 8 are plots of simulated electrophysiological recordings obtained using the nanoparticle probes according to an embodiment of the present disclosure.

### DETAILED DESCRIPTION

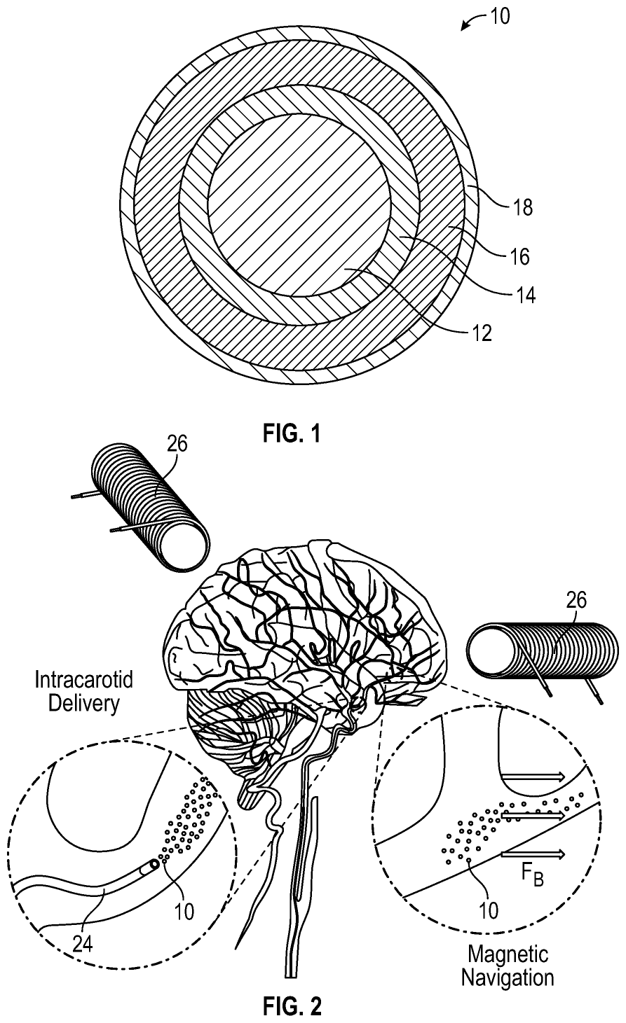

With reference to FIG. 1, a nanoparticle probe 10 according to the present disclosure includes a core 12, which may be formed from a dielectric material, such as silica (SiOₓ). In embodiments, a semiconductive material (i.e., silicon, germanium, gallium, arsenide, and variants thereof, such as alloys, oxides, and nitrides) may be used in lieu of or in addition to the dielectric material. The core 12 may also be formed from magnetic material, such as magnetite (Fe₃O₄). The core 12 may have a substantially spherical shape and may have a diameter from about 80 nm to about 150 nm, and in embodiments, may be from about 100 nm to about 130 nm. The diameter of the core 12 may be selected to exhibit resonance scattering at a specific wavelength of light being used to illuminate the nanoparticle probe 10, e.g., NIR light having a wavelength of 1000 nm or above, as described in further detail below.

The core 12 may be formed using any suitable method for producing spherical nanoparticles. With respect to magnetic nanoparticles, in order to achieve monodisperse batches. The ratio of stabilizers, temperature, and stirring may be adjusted to control the size, shape, and crystallinity during the formation of magnetite particles. Relatively uniform distributions for both dielectric and magnetic cores 12 may be achieved, and a more uniform distribution of particles may be obtained by iteratively centrifuging, pelleting, and resuspending particles at different speeds. Larger particles may be separated in pelleting and may be resuspended in another solution usually brief sonication.

The nanoparticle probe 10 also includes a conductive shell 14 disposed over the core 12. The conductive shell 14 may be formed from a conductive material, such as graphene and/or metals, including but not limited to, gold, silver, aluminum, copper, titanium, magnesium, palladium, zirconium, and variants thereof, such as alloys, oxides, and nitrides. The conductive shell 14 may have a thickness from about 1 nm to about 10 nm, and in embodiments, may be about 5 nm.

The conductive shell 14 may be formed in a similar manner on both the dielectric and magnetic cores 12 except for a functionalization step for initially seeding the conductive material (i.e., gold). Dielectric cores 12 may be disposed in a first solvent, which may be ethanol, and functionalized by (3-Aminopropyl)triethoxysilane (APTES). Similarly, the magnetic cores 12 may be dispersed in a solvent, but are functionalized using (3-Aminopropyl)trimethoxysilane (APTMS) instead of APTES. After functionalization, the cores 12 are silanized allowing for formation of the conductive shell 14. Excess supernatant may be removed by centrifugation and resuspension in ethanol.

The conductive shell 14 may be formed by attaching precursor conductive nanoparticles having a diameter from about 1 nm to about 5 nm to the silanized core 12. Thus, the conductive nanoparticles may be used to seed the precursor for the conductive shell 14. The precursor conductive nanoparticles may be stabilized in a second solvent, which may be tetrakis(hydroxymethyl)phosphonium chloride (THPC). The precursor conductive nanoparticles may be sonicated with the silanized cores 12 and a salt solution for a first period of time, which may be from 1 minute to about 10 minutes, left for a second period to reach equilibrium, which may be from 8 hours to about 16 hours. The solution with the seeded cores 12 may then centrifuged, the supernatant is then removed, and the cores 12 are resuspended with a sonicating probe in water. After the cores 12 are centrifuged and resuspended again in water, the precursor particles are mixed with a plating solution of the conductive material and aerated by carbon monoxide (CO). This reduces the seeded conductive material (i.e., the precursor conductive nanoparticles) and forms the conductive shell 14. The thickness of the conductive shell 14 may be adjusted by controlling the aeration time.

The nanoparticle probe 10 further includes an electrochromic polymer coating 16 disposed over the conductive shell 14. The electrochromic polymer coating 16 is biocompatible and does not exhibit cytotoxicity. Suitable biocompatible electrochromic polymers for forming the electrochromic polymer coating 16 include, but are not limited to, poly(3,4-thylenedioxythiophene):polystyrene sulfonate (PEDOT:PSS), polypyrrole, polyaniline, poly(3,4-propylenedioxythiophene) (polypro DOT), combinations and derivatives thereof. The electrochromic polymer coating 16 may have a thickness from about 10 nm to about 30 nm, and in embodiments, may be about 15 nm.

The electrochromic polymer coating 16 may be formed by placing the cores 12 in a solution of the electrochromic polymer. The cores 12 may be functionalized prior to being placed in the solution. In embodiments, a PEDOT:PSS coating may be applied to the conductive shell 14 by initially mixing the cores 12 having the conductive shell 14 in 3,4-ethylene-dioxythiophene (EDOT) in hydrochloric acid and sodium dodecylsulfate (SDS) while stirring. This mixture may then be added to an ammonium persulfate solution and stored for approximately 24 hours or more. The solution may then be centrifuged and redispersed in SDS for further use. According to another embodiment, the conductive shell 14 of the cores 12 may be functionalized with thiophene using APTES. The PEDOT:PSS coating may then be subsequently covalently bonded to the cores 12 when they are dispersed in dodecylbenzene sulfonic acid (DBSA), the monomer, EDOT, and iron (III) chloride (FeCl₃) and stirred for approximately 24 hours. After this, the coated cores 12 and 22 are washed in deionized water and ethanol for further use. These coating methods may be applied to the cores 12 after functionalizing the conductive shell 14 with thiophene or other thiols.

The nanoparticle probe 10 may have a generally spherical shape having a diameter from about 140 nm to about 200 nm. As the ratio of materials used to form the nanoparticle probe 10 ultimately determines its spectrum, the total particle diameter is determined by the tradeoff between SSNR and permeability in the subject larger particles have more limited mobility, which affects transport across the BBB and within the interstitial space.

The nanoparticle probe 10 is configured to exhibit resonance scattering at a specific wavelength. Parameters which affect resonance scattering include size and shape of the core 12 and thickness of the conductive shell 14. Thus, in embodiments where the core 12 is formed from silica having a radius of about 63 nm and the conductive shell 14 is formed of gold having a thickness of about 5 nm, the nanoparticle probe 10 exhibits resonance scattering from about 1000 nm to about 1100 nm, and in embodiments about 1050 nm, which corresponds to a wavelength regime enabling deep tissue penetration. Various modifications to the dimensions and material properties are envisioned to achieve resonance scattering at specific wavelengths of NIR II light.

A spherically symmetric structure of the nanoparticle probe 10 and the core 12 minimize polarization dependence. A plasma frequency modulation of Δωₚ=(ωₚ/2N)ΔN is a result of surface charge density variation ΔN due to a transient external field. Unlike in vitro electro-plasmonic probes, the nanoparticle probes 10 are designed for (i) electro-plasmonic operability at the infrared frequencies and (ii) colloidal core-shell structure for solution-based delivery and non-invasive detection of the electrophysiological signals.

In further embodiments, the nanoparticle probe 10 may also include an optional biological coating 18 to prevent rejection by biological tissue since without a biological coating the nanoparticle probe 10 may be quickly cleared from the body or develop a protein corona inhibiting cellular attachment. Additionally, a functional coating may be used to enhance transport across the blood-brain barrier (BBB). The biological coating 18 may be formed from biomolecules, such as lipids, proteins, peptides, etc. which can be adsorbed or covalently bonded to conductive polymers of the electrochromic polymer coating 16. While a wider variety of functionalization is available for adsorption, the biological coating 18 may degrade over time in serum and is thus more useful in shorter duration use. Covalent bonding of biomolecules may be better suited for long duration cell adhesion after crossing the BBB, but physisorption may be sufficient for simply crossing the BBB. In embodiments, peptides may be attached to PEDOT and PEDOT:PSS, which is more reactive due to its sulfonic groups and carboxyl groups are used as a bridge to adhere peptides to the surface. Nanoparticle design considerations in bypassing the BBB for intracarotid delivery are the most relevant for whole brain imaging whereas nanoparticle probes 10 which are directly introduced to the cerebrospinal fluid (CSF) may not need further functionalization.

The nanoparticle probes 10 according to the present disclosure may be used for optical sensing of electric fields, specifically for sensing extracellular field produced by neurons. Thus, the nanoparticle probes 10 are initially delivered to the brain to enable sensing. The nanoparticle probes 10 may be delivered to the brain parenchyma (i.e., extracellular space of glia and neurons) in a variety of ways, including, but not limited to, intracarotid delivery, CSF delivery, intranasal delivery, and the like. Prior to delivery, the nanoparticle probes 10 may be placed in a saline solution or any other suitable biocompatible fluid for storing and injecting the nanoparticle probes 10.

CSF delivery may be accomplished using cerebellomedullary cistern delivery, which bypasses the BBB through the CSF and allows for use of the nanoparticle probes 10 in the brain without surface functionalization. Subjects may be anesthetized and placed in a stereotactic frame, but without need for invasive surgery beyond a simple injection. The nanoparticle probes 10 diffuse throughout the subarachnoid space (SAS) and can access the deep parenchyma of the brain through the Virchow-Robin space. Permeation throughout the brain may depend on particle size of the nanoparticle probes 10 since particles having a diameter larger than 100 nm injected into the cisterna magna are largely limited to the SAS.

CSF delivery may also be accomplished using intrathecal injections which enter the same fluid space but from the lumbar region. CSF delivery methods may be used for examining neurons on the outer surface of the brain or along the spinal cord as there is no need for surface functionalization of the nanoparticle probes 10 except to add specificity in neural attachment.

Delivery through the internal carotid artery may include minimally invasive surgery and incorporating functionalized nanoparticle coatings to overcome the BBB. A local anesthetic is administered to the subject and a small incision is formed such that the entire process may be done while they are awake. As shown in FIG. 2, a microcatheter 24 is then inserted through the incision and the nanoparticle probes 10 are injected into the artery. One or more magnets 26 may be used to guide the nanoparticle probes 10 in embodiments where the cores 12 are formed from a magnetic material. The magnets 26 may generate magnetic field of varying strength and directionality allowing for moving the nanoparticle probes 10 from the injection site to a desired location within the brain and maintaining their position therein. The magnets 26 may be either permanent or electromagnets configured to generate a sufficiently strong magnetic field for affecting the nanoparticle probes 10.

Using this delivery method consideration may be given to the type and amount of functionalization of the nanoparticle probes 10. The biological coating 18 may include lipids, proteins targeting insulin, transferrin, low density lipoprotein (LDL), or other receptors configured to induce transcytosis. After applying the biological coating 18, the nanoparticle probes 10 may be pelleted by centrifugation, suspended in water, and sonicated briefly to obtain an injectable solution of nanoparticle probes 10. In embodiments, mannitol may be injected to supplement preparation of receptor-mediated transcytosis with chemically enhanced permeability by temporarily boosting the permeability of the BBB. Subsequently, the nanoparticle probes 10 may be delivered via intracarotid injection where they cross the BBB and diffuse through the parenchymal space. In embodiments, intracarotid injection may be direct, i.e., using a syringe, without using the microcatheter 24 and may be done at an infra-ophthalmic location within the intracarotid artery and at an approximate rate ≥17 ml/min to avoid streaming and ensuring that the nanoparticle probes 10 are dispersed into capillaries.

Other delivery methods may also be used to bypass the BBB such as intraventricular injection, while this method is as effective as cerebellomedullary cistern delivery, it involves the invasive implantation of a ventricular catheter and a deep injection which breaks the skull and scars brain parenchyma.

In further embodiments, the nanoparticle probes 10 may be injected into the brain via stereotactic injection with a high degree of localization to the region of interest. This may be done with the aid of imaging systems, such as functional magnetic resonance imaging (fMRI) to locate symptomatic regions and without surface functionalization on the nanoparticle probes 10. This delivery method involves making a burr hole the skull and using a needle entry, which may subsequently scar brain tissue during injection. However, this is still much less traumatic than currently available brain activity monitoring methods, such as those using iEEG.

An intranasal delivery method may also be used since the olfactory bulb provides near direct access to the CSF for nanoparticle probes 10, thus no surface functionalization would be needed. Additionally, it is a less invasive method by not requiring incisions or placing subjects in stereotactic frames. However, since very little fluid (e.g., approximately 1 mL) can be accepted with this method, the delivery fluid has to have a suitable concentration of the nanoparticle probes 10.

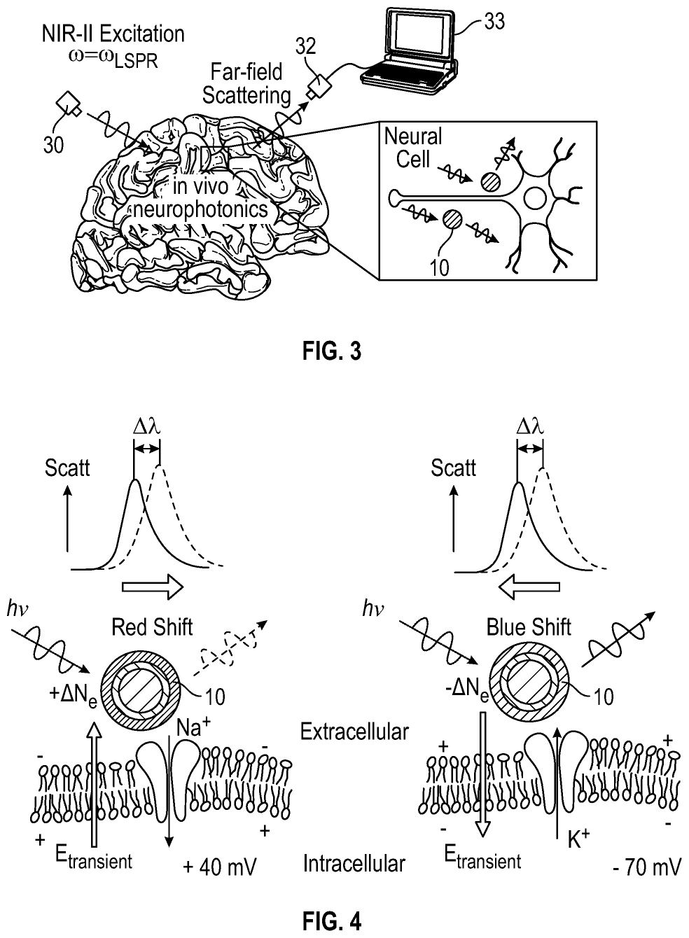

With reference to FIGS. 3 and 4, the nanoparticle probe 10 according to the present disclosure is configured to directly measure local electric-field dynamics (e.g., neural depolarization events) through NIR light. More specifically, the nanoparticle probe 10 uses two fundamental mechanisms for electro-optic translation: (1) localized surface plasmon (LSP) enhanced scattering cross-section, and (2) drastic electro-optic sensitivity to local electric-field dynamics through the electrochromic loading of the electrochromic polymer coating 16.

FIG. 3 depicts the proposed swarm-and-lock concept schematically and a system for using the nanoparticle probes 10 for monitoring neural activity. Nanoparticle probes 10 are first distributed within the cortical regions of the brain as described above using different delivery methods. In addition, the nanoparticle probes 10 may be functionalized using specific proteins to tether the nanoparticle probes 10 to specific cell membranes through the functionalized proteins. Once at the target site, a light source 30 configured to illuminate the target site with NIR-II light at a wavelength from about 1,000 nm to about 1,700 nm, which penetrates tissue and is backscattered by the nanoparticle probes 10. An imaging sensor 32 is configured to receive backscattered NIR-II light. The imaging sensor 32 is coupled to a computing device 33 configured to output (e.g., on a display) images corresponding to the backscattered NIR-II light. In embodiments, a first color (e.g., green) may be used to represented backscattered light from the nanoparticle probes 10 during depolarization and a second color (e.g., yellow) may be used to represent backscattered light during hyperpolarization. Thus, far-field scattering signal from the nanoparticle probes 10 may be employed to monitor the neural activity, that is the transient electric field created by a discharging neuron since the changes in the electrical fields result in a wavelength shift of the backscattered NIR-II light.

With reference to FIG. 4, the nanoparticle probe 10 is disposed in proximity of a neuron such that the nanoparticle probe 10 is affected by the changes in the ionic current and/or electrical fields of the neuron. During depolarization events, the membrane potential is controlled by the ionic current, that is sodium (Na⁺) and potassium (K⁺) movement between the inside and outside of the cell. Large fluctuations in the membrane potential occur as a result of Na⁺ influx into the cell (spike or depolarizing phase) and K⁺ efflux from the cell (hyperpolarizing phase). Cell depolarization (spiking) causes a high transient electric field leading to increased light scattering and red shifting of the electro-plasmonic (electrochromic-plasmonic) nanoantenna resonance spectrum. A return to resting potential (hyperpolarization) results in reversal of the scattering spectrum changes.

Such large charge density (ion concentration) perturbations give rise to strong transient (pre-screening) electric fields, Eₜᵣₐₙₛᵢₑₙₜ. Thus, the strength of Eₜᵣₐₙₛᵢₑₙₜ outside the cellular membrane may be expressed as a charge transfer model. A neural cell membrane can be treated as a 20 μm diameter spherical lipid bilayer. The specific capacitance of this cellular membrane is approximately Cₘ=1 μF/cm². Considering a transmembrane potential variation of approximately 110 mV, the total charge that is moved across a cell membrane during a spiking event may be calculated using this capacitance in formula (I):

$$Δ Q = {C}_{m} {A}_{cell} Δ {V}_{m} . \qquad ( I )$$

In formula (I), A_{cell} is the total surface area of the cellular membrane and ΔVₘ is the change in the membrane potential. This charging event may include approximately 8.6 million monovalent Na⁺ ions rushing into the cell during the spiking phase. Thus, instantaneous extracellular electric field strength right outside the cell may be calculated using this extra charge and the dielectric constant (ε_{CSF} is about 88.9) of the cerebrospinal fluid. This model accurately captures the extracellular field values, which are typically few tens of mV/nm in strength. Such large extracellular electric fields may lead to strong modulations in the backscattering of the nanoparticle probes 10, enabling remote detection of electrical fields due to neural activity, as illustrated in FIG. 4.

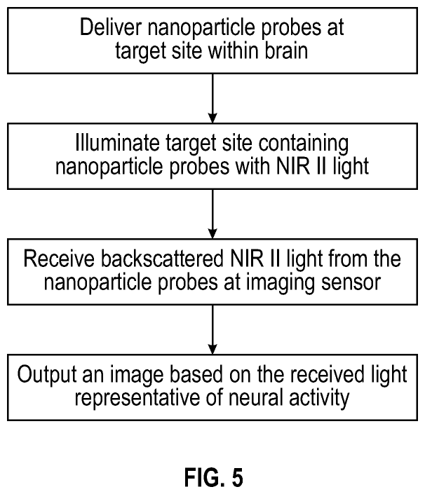

With reference to FIG. 5, a method for imaging neural activity using the nanoparticle probes 10 includes delivering the nanoparticle probes 10 to a target site within the brain using any suitable delivery method, such as those described above. Delivery of the nanoparticle probes 10 may be monitored using NIR-II light provided by the light source 30, which may be used to illuminate the implantation site and the target site since the nanoparticle probes 10 scatter NIR-II light regardless of their location within the body. The scattered light is received at the imaging sensor 32, and the computing device 33 may then convert the received scattered right to a representative color for display.

Once the nanoparticle probes 10 are at the target site, the light source 30 is used to illuminate the target site with the nanoparticle probes 10 therein. The nanoparticle probes 10 backscatter the NIR-II light, which shifts based the changes in the electrical fields due to neural activity. The backscattered light is received at the imaging sensor 32 which is coupled to the computing device 33 configured to output visible colors, e.g., blue and red, corresponding to the backscattered light. The changes in light correspond to the blue and red shifts in the scattered NIR-II light by the nanoparticle probes 10, which is in turn, reflective of neural activity. Thus, neural activity may be monitored in real time by observing and/or recording changes in color through the computing device 33.

The following Examples illustrate embodiments of the present disclosure. These Examples are intended to be illustrative only and are not intended to limit the scope of the present disclosure.

### Example 1

This Example describes optical scattering efficiency of the nanoparticle probes according to the present disclosure.

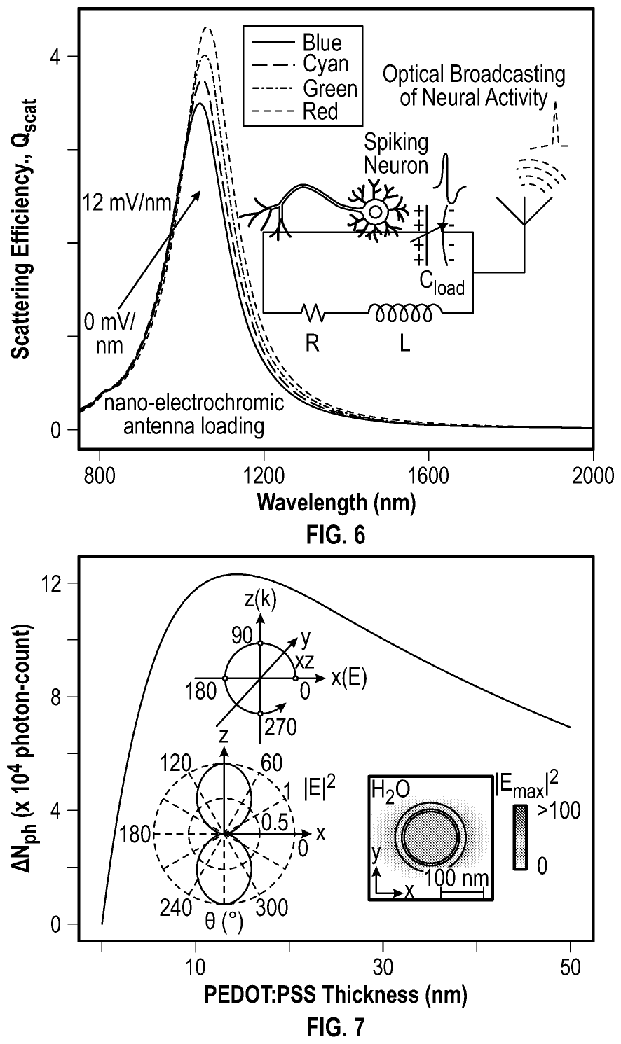

FIG. 6 shows optical scattering efficiency, Q_{scat}, spectra for silica-gold nanoshells loaded with PEDOT:PSS for varying electric field strengths (0-12 mV/nm) with increments of 4 mV/nm. A core-shell nanoparticle with 63 nm silica core radius, 5 nm thick gold shell, and 15 nm thick PEDOT: PSS coating was used for this experiment. Inset shows an equivalent “lumped” nanocircuit model of the nanoparticle probes 10 at optical frequencies. Local electric-field dynamics is translated to nanoantenna resonance frequency modulation through capacitive (PEDOT:PSS) loading effects.

The scattering efficiency was calculated for electric field strengths ranging from about 0 mV/nm to about 12 mV/nm with increments of about 4 mV/nm. Nanoparticle probes 10 demonstrated a scattering efficiency modulation over 20% for an electric field of 12 mV/nm at LSP resonance wavelength at approximately 1050 nm. Electro-optic modulation up to about 40% were also shown at longer wavelengths. The electro-optic response of the nanoparticle probes 10 was analyzed using a lumped optical nanocircuit model for the electrochromic polymer (PEDOT)-plasmonic nanoantenna system. The PEDOT electrochromic load, acting as an electric field-controlled nano-capacitor, C_{load}, translates the electric-field dynamics of the neural cell to the scattering signal modulation (inset of FIG. 6).

### Example 2

This Example describes differential scattering of the nanoparticle probes according to the present disclosure with varying PEDOT:PSS load thickness.

FIG. 7 shows differential scattering signal plots for nanoparticle probes 10 with varying PEDOT:PSS load thickness. The polar plot of angular scattering by the nanoprobe for linearly polarized incident light (1050 nm) in the x direction is shown in the bottom left inset. The top inset illustrates how the scattering angle is defined within the xz plane. Electromagnetic “hotspots” due to plasmonic field enhancement at 1050 nm are shown in the bottom right inset. Three-dimensional finite difference time domain (FDTD) calculations were employed to calculate angular scattering and cross-sectional field profile.

Dependance of the scattering signal on PEDOT:PSS layer thickness was analyzed (FIG. 7). Conventionally, thicker electrochromic films are used to achieve a strong differential signal, which results in slower temporal response. Nanoparticle probes 10 realizes faster response times and strong signal modulations simultaneously using enhanced light-matter interactions in nanoscale electromagnetic ‘hotspots’ around the plasmonic core-shell structure (FIG. 7, bottom right inset). Sub-millisecond switching times down to approximately 200 us are achievable using a PEDOT layer having a thickness of about 20 nm on lithographically fabricated electro-plasmonic nanodisk antenna. The differential optical signal (photon count) was calculated using formula (II):

$$Δ {N}_{ph} = {I}_{inc} ( Δ {Q}_{sca} π {r}^{2} ) ( λ / hc ) η {Tt}_{int} \qquad ( II )$$

where ΔQ_{sca} is the change in scattering cross section, line is the incident light intensity (10 mW/mm²), η is the solid angle fraction of the total scattered light collected by a microscope objective (assuming a 20× obj., NA=0.9) [16]), T is the detection efficiency (quantum yield 0.5), tᵢₙₜ is the integration time (1 ms), c is the speed of light, h is Planck's constant, r is the radius of the Nanoparticle probes 10, and λ is the probing wavelength. As shown in FIG. 7, a single Nanoparticle probes 10 loaded with a thin layer of 5-20 nm PEDOT:PSS was able to generate a large differential signal (approximately 120 k photons) that were be readily detected. Decreasing scattering signal with increasing PEDOT:PSS thickness beyond approximately 20 nm was due to the de-tuning of the electro-plasmonic resonance and probing wavelength of about 1050 nm with increasing dielectric loading.

### Example 3

This Example describes signal to noise ratio measurements.

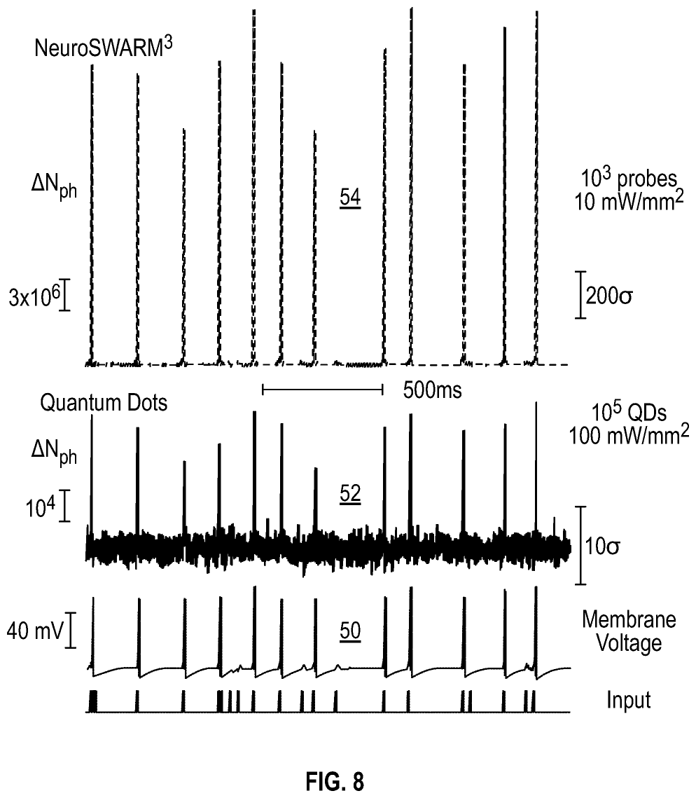

FIG. 8 shows simulated electrophysiological recordings for nanoparticle probes 10 and quantum dots (QDs). A phasic spiking Izhikevich model ws used for the neural spiking activity at pseudo-random times. The input and resulting membrane voltage are shown in a bottom plot 50. Differential fluorescence signal from 10⁵ CdSe quantum dots are shown in a middle plot 52 for an illumination intensity of 100 mW/mm² light for a maximum extracellular field of 3 mV/nm. Differential scattering signals obtained from 10³ Nanoparticle probes 10 probes (top plot 54) is shown assuming a light intensity of 10 mW/mm² light at 1050 nm. Scales indicating the photon count for the differential signal and the standard deviation due to the shot noise are shown on the left and right, respectively.

The functionality of the nanoparticle probes 10 was demonstrated for label-free optical detection of depolarization events using an Izhikevich model for a neuron. Constant voltage pulses (1 mV, 10 ms) were provided as an input at pseudo-random times such that inputs averaged 10 Hz, and model parameters were set such that the neuron exhibits phasic spiking (FIG. 8, plot 50). Results were generated in 0.1 ms steps and data was compressed by taking the maximum every ten points to preserve relative spike amplitudes for 1 ms integration times. The extracellular field was largely determined by the flow of ions across the cell membrane during spiking. At rest, an ionic double layer forms at the surface of the membrane and screens nearly all electric field. However, strong transient electric fields are created during the depolarization events before screening layers is established as described above. As the cell membrane acts as a discharging capacitor, this field was approximated by taking the derivative of membrane voltage with respect to time, I∝dV/dt and adjusting the amplitude of the resulting field to 3 mV/nm.

In FIG. 8, the differential signal of the nanoparticle probes 10 was compared with that of CdSe quantum dots (QDs), which have recently received significant attention as a high photon count alternatives to GEVIs. Differential fluorescence provided by a QD due to an external electric field can be expressed as in formula (III):

$$Δ F / {F}_{0} = - {Δτ}_{r} / {τ}_{r} ( 1 - {Φ}_{F} ) \qquad ( III )$$

where Φ_{F}=0.5 is the quantum efficiency and Δτᵣ/τᵣ=0.5% is the percentage change in the fluorescence lifetime of decaying excitations. QDs, that have a 1.2 nm² cross section, are illuminated with 100 mW/mm² visible light (650 nm). Resulting traces show that individual Nanoparticle probes 10 probes (FIG. 8, plot 54) readily outperform QDs (FIG, plot 52) by providing at least four orders of magnitude higher photon count measurements (left axis), despite the use of 10-fold reduced light intensity (10 mW/mm²). The fundamental detection limit to any optical measurement technique is the shot noise limit SSNR which may be expressed by formula (IV):

$$( Δ S / {S}_{0} ) √ {N}_{ph} \qquad ( IV )$$

where ΔS/S₀ is the differential scattering signal and Nₚₕ is the photon count. As shown in FIG. 8, a drastically high SSNR measurement capability (SSNR˜10³) was also noted for the nanoparticle probes 10 with respect to QDs (right axis).

It will be appreciated that of the above-disclosed and other features and functions, or alternatives thereof, may be desirably combined into many other different systems or applications. Also, that various presently unforeseen or unanticipated alternatives, modifications, variations, or improvements therein may be subsequently made by those skilled in the art which are also intended to be encompassed by the following claims. Unless specifically recited in a claim, steps, or components according to claims should not be implied or imported from the specification or any other claims as to any particular order, number, position, size, shape, angle, or material.

What is claimed is:

1. A nanoparticle probe for detecting neural activity, the nanoparticle probe comprising:
a core having a substantially spherical shape;
a conductive shell disposed over the core; and
an electrochromic polymer coating disposed over the conductive shell.

2. The nanoparticle probe according to claim 1, further comprising a biological coating including at least one of lipids, proteins, or peptides integrated with or disposed over the electrochromic polymer.

3. The nanoparticle probe according to claim 1, wherein the core is formed from a dielectric material or a magnetic material.

4. The nanoparticle probe according to claim 1, wherein the core includes at least one of silica or magnetite.

5. The nanoparticle probe according to claim 1, wherein the core has a diameter from about 80 nm to about 150 nm.

6. The nanoparticle probe according to claim 1, having a diameter from about 140 nm to about 200 nm.

7. The nanoparticle probe according to claim 1, wherein the conductive shell includes at least one of graphene, gold, silver, aluminum, copper, titanium, magnesium, palladium, and zirconium.

8. The nanoparticle probe according to claim 1, wherein the electrochromic polymer coating includes at least one of poly(3,4-ethylenedioxythiophene): polystyrene sulfonate, polypyrrole, polyaniline, or poly(3,4-propylenedioxythiophene).

9. The nanoparticle probe according to claim 1, wherein the conductive shell has a thickness from about 1 nm to about 10 nm.

10. The nanoparticle probe according to claim 1, wherein the electrochromic polymer coating has a thickness from about 10 nm to about 30 nm.

11. The nanoparticle probe according to claim 1, the core and the conductive shell are configured to exhibit resonance scattering of near infrared light having a wavelength from about 1000 nm to about 1100 nm.

12. A method for monitoring neural activity, the method comprising:
delivering a plurality of nanoparticle probes to a target site within a brain;
illuminating the target site with a near infrared light having a wavelength from about 1000 nm to about 1700 nm;
receiving backscattered light from the plurality of nanoparticles illuminated by the near infrared light; and
determining neural activity based on the backscattered light from the plurality of nanoparticles.

13. The method according to claim 12, wherein delivering includes at least one of cerebellomedullary cistern delivery, intracarotid delivery, intraventricular injection, stereotactic brain injection, or intranasal delivery.

14. The method according to claim 12, wherein the plurality of nanoparticle probes is configured to shift their backscattering spectrum in response to a change in an electrical field at the target site.

15. The method according to claim 12, wherein each nanoparticle probe of the plurality of probes includes:
a core having a substantially spherical shape;
a conductive shell disposed over the core; and
an electrochromic polymer coating disposed over the conductive shell.

16. The method according to claim 15, wherein each nanoparticle probe of the plurality of probes further includes a biological coating including at least one of lipids, proteins, or peptides.

17. The method according to claim 15, wherein the core is formed from a magnetic material.

18. The method according to claim 16, wherein delivering includes magnetically guiding the plurality of nanoparticle probes to the target site.

19. A system for monitoring neural activity, the system comprising:
a light source configured to illuminate a target site in a brain with a near infrared light having a wavelength from about 1000 nm to about 1700 nm;
a plurality of nanoparticle probes disposed at the target site, each of the nanoparticle probes includes:
a core having a substantially spherical shape;
a conductive shell disposed over the core; and
an electrochromic polymer coating disposed over the conductive shell;
an image sensor configured to receive backscattered light from the plurality of nanoparticles illuminated by the near infrared light;
wherein the plurality of nanoparticle probes is configured to shift their backscattering spectrum in response to a change in an electrical field at the target site.

20. The system according to claim 19, further comprising a biological coating including at least one of lipids, proteins, or peptides.

21. The system according to claim 19, wherein the core is formed from a dielectric material or a magnetic material.

22. The system according to claim 19, wherein the core includes at least one of silica or magnetite.

23. The system according to claim 19, wherein the core has a diameter from about 80 nm to about 150 nm.

24. The system according to claim 19, wherein each of the nanoparticle probes has a diameter from about 140 nm to about 200 nm.

25. The system according to claim 19, wherein the conductive shell includes at least one of graphene, gold, silver, aluminum, copper, titanium, magnesium, palladium, and zirconium.

26. The system according to claim 19, wherein the electrochromic polymer coating includes at least one of poly(3,4-ethylenedioxythiophene): polystyrene sulfonate, polypyrrole, polyaniline, or poly(3,4-propylenedioxythiophene).

27. The system according to claim 19, wherein the conductive shell has a thickness from about 1 nm to about 10 nm.

28. The system according to claim 19, wherein the conductive shell has a from about 10 nm to about 30 nm.

29. The system according to claim 19, the core and the conductive shell are configured to exhibit resonance scattering of near infrared light having a wavelength from about 1000 nm to about 1100 nm.
